(19) United States
(12) Patent Application Publication
(10) Pub. No.: US 2024/0304330 A1
(43) Pub. Date: Sep. 12, 2024

(54) SYSTEM AND METHOD FOR TRAINING ARTIFICIAL INTELLIGENCE MODELS USING SUB-GROUP TRAINING DATASETS OF A MAJORTIY CLASS OF SAMPLES

(71) Applicant: GE Precision Healthcare LLC, Waukesha, WI (US)

(72) Inventors: Hariharan RAVISHANKAR, Bangalore (IN); Rohan Keshav PATIL, Bangalore (IN); Prasad Sudhakara MURTHY, Bangalore (IN)

(21) Appl. No.: 18/597,458

(22) Filed: Mar. 6, 2024

Related U.S. Application Data
(60) Provisional application No. 63/488,669, filed on Mar. 6, 2023.

(51) Int. Cl. G16H 50/20; G16H 10/60

(52) U.S. Cl. CPC G16H 50/20; G16H 10/60

(57) ABSTRACT

Various systems and methods are provided for training and using a diagnostic model including artificial intelligence (AI) models. The diagnostic model including the AI models may be trained by receiving training data including a majority class of samples corresponding to medical data of patients that do not have the medical condition and a minority class of samples corresponding to medical data of patients that do have the medical condition, determining sub-groups of the majority class of samples based on features of the majority class of samples, generating sub-group training datasets that each include respective samples of the sub-groups of the majority class of samples and samples of the minority class of samples, and training the AI models of the diagnostic model using the sub-group training datasets. The diagnostic model including the AI models may be used to determine whether a patient has a medical condition.

### CROSS-REFERENCE TO RELATED APPLICATION(S)

This patent application claims the benefit of priority to U.S. Provisional Application No. 63/488,669, filed on Mar. 6, 2023, the entirety of which is incorporated herein by reference.

### TECHNICAL FIELD

The present disclosure relates, generally, to systems and methods for training artificial intelligence (AI) models using training datasets that reduce an imbalance between a majority class of samples and a minority class of samples. More specifically, the present disclosure relates to systems and methods for training AI models using sub-group training datasets of a majority class of samples, and for training AI models using balanced training datasets that are more balanced that an imbalanced training dataset. Moreover, the present disclosure relates to systems and methods for using AI models of a diagnostic model to determine whether a patient has a medical condition.

### BACKGROUND

Ensemble learning may refer to an AI technique that utilizes the outputs from multiple AI models to determine a prediction. For instance, in the medical domain, a diagnostic model may include several constituent AI models that each provide a determination of whether a patient has a medical condition. The diagnostic model may determine whether the patient has the medical condition based on the respective outputs of the constituent AI models.

A training dataset may include a majority class of samples and a minority class of samples. The majority class of samples may be medical data of patients that do not have the medical condition, and the minority class of samples may be medical data of patients that do have the medical condition. In this way, the interest is typically in the minority class of samples because the minority class of samples represents medical conditions that clinicians would like to preemptively predict. However, training datasets are usually “imbalanced” in that the training datasets include a vastly greater number of samples for the majority class than as compared to the number of samples for the minority class.

In some cases, respective AI models of a diagnostic model are trained using the imbalanced training dataset. In these cases, the trained AI models and diagnostic model might not accurately predict whether a patient has a particular medical condition seen in the minority class of samples. In other cases, techniques to address the imbalance concern, such as “under-bagging,” may be performed. In these cases, generating AI models using such techniques might result in a large number of AI models in the ensemble, which increases memory consumption, increases processor consumption, increases training time, etc.

### SUMMARY

This summary introduces concepts that are described in more detail in the detailed description. It should not be used to identify essential features of the claimed subject matter, nor to limit the scope of the claimed subject matter.

In an aspect, a method may include receiving medical data of a patient; determining whether the patient has a medical condition using the medical data and a diagnostic model including artificial intelligence (AI) models; and transmitting or displaying information identifying the determination of whether the patient has the medical condition, wherein the diagnostic model including the AI models is trained by: receiving training data including a majority class of samples corresponding to medical data of patients that do not have the medical condition and a minority class of samples corresponding to medical data of patients that do have the medical condition, determining sub-groups of the majority class of samples based on features of the majority class of samples, generating sub-group training datasets that each include respective samples of the sub-groups of the majority class of samples and samples of the minority class of samples, and training the AI models of the diagnostic model using the sub-group training datasets.

In another aspect, a device may include a memory configured to store instructions; and one or more processors configured to execute the instructions to perform operations comprising: receiving medical data of a patient; determining whether the patient has a medical condition using the medical data and a diagnostic model including artificial intelligence (AI) models; and transmitting or displaying information identifying the determination of whether the patient has the medical condition, wherein the diagnostic model including the AI models is trained by: receiving training data including a majority class of samples corresponding to medical data of patients that do not have the medical condition and a minority class of samples corresponding to medical data of patients that do have the medical condition, determining sub-groups of the majority class of samples based on features of the majority class of samples, generating sub-group training datasets that each include respective samples of the sub-groups of the majority class of samples and samples of the minority class of samples, and training the AI models of the diagnostic model using the sub-group training datasets

In yet another aspect, a non-transitory computer-readable medium may store instructions that, when executed by one or more processors, cause the one or more processors to perform operations comprising: receiving medical data of a patient; determining whether the patient has a medical condition using the medical data and a diagnostic model including artificial intelligence (AI) models; and transmitting or displaying information identifying the determination of whether the patient has the medical condition, wherein the diagnostic model including the AI models is trained by: receiving training data including a majority class of samples corresponding to medical data of patients that do not have the medical condition and a minority class of samples corresponding to medical data of patients that do have the medical condition, determining sub-groups of the majority class of samples based on features of the majority class of samples, generating sub-group training datasets that each include respective samples of the sub-groups of the majority class of samples and samples of the minority class of samples, and training the AI models of the diagnostic model using the sub-group training datasets.

### BRIEF DESCRIPTION OF DRAWINGS

FIG. 1 is a diagram of a system for training and using AI models of a diagnostic model.

FIG. 2 is a diagram of example components of one or more devices of the system of FIG. 1.

FIG. 3 is a flowchart of an example process for training AI models of a diagnostic model using sub-group training datasets.

FIG. 4 is a diagram of example training data.

FIG. 5 is a flowchart of an example process for determining sub-groups of a majority class of samples based on features determined in clinical metadata.

FIG. 6 is a flowchart of an example process for determining sub-groups of a majority class of samples based on features extracted from training data.

FIG. 7 is a diagram of example sub-group training datasets.

FIG. 8 is a diagram of training AI models of a diagnostic model using sub-group training datasets.

FIG. 9 is a flowchart of an example process for determining whether a patient has a medical condition using a diagnostic model including AI models trained using sub-group training datasets.

FIG. 10 is a flowchart of an example process for training AI models of a diagnostic model using balanced training datasets.

FIG. 11 is a diagram of example imbalanced training data.

FIG. 12 is a diagram of example balanced training datasets.

FIG. 13 is a diagram of training AI models of a diagnostic model using balanced training datasets.

FIG. 14 a flowchart of an example process for determining whether a patient has a medical condition using a diagnostic model including AI models trained using balanced training datasets.

FIG. 15 is a diagram of an example process for training, deploying, and monitoring a diagnostic model.

### DETAILED DESCRIPTION

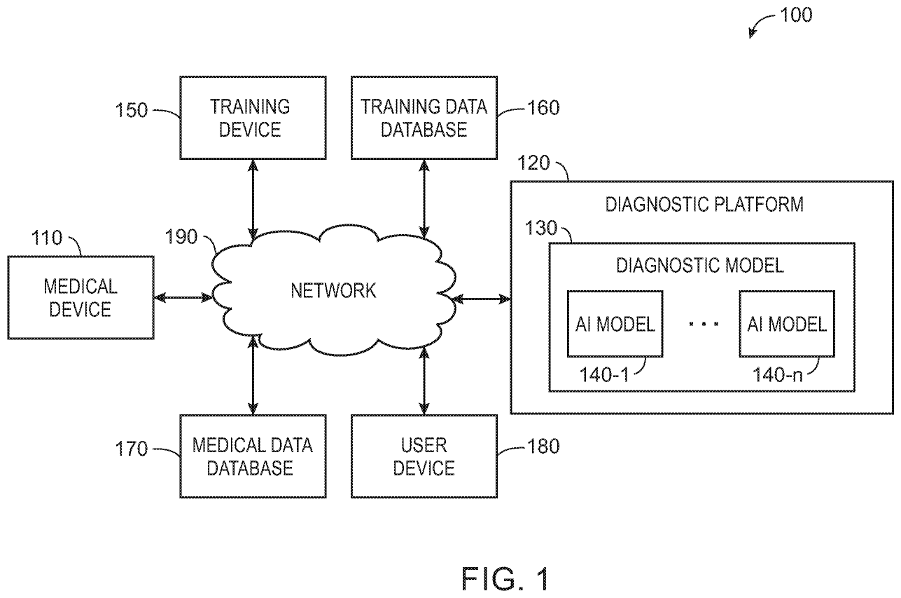

FIG. 1 is a diagram of a system 100 for training and using AI models of a diagnostic model. As shown in FIG. 1, the system 100 may include a medical device 110, a diagnostic platform 120, a diagnostic model 130, AI models 140-1 through 140-n, a training device 150, a training data database 160, a medical data database 170, a user device 180, and a network 190.

The medical device 110 may be configured to generate medical data of a patient. For example, the medical device 110 may be an electrocardiogram (ECG) device, an electroencephalogram (EEG) device, an ultrasound device, a magnetic resonance imaging (MRI) device, an X-ray device, a computed tomography (CT) device, or the like. It should be understood that the embodiments herein are applicable to any type of medical data generated by any type of medical device.

The diagnostic platform 120 may be configured to determine whether a patient has a medical condition using the AI models 140-1 through 140-n. For example, the diagnostic platform 120 may be a server, a computer, a virtual machine, or the like. The diagnostic model may be a model configured to determine, using medical data of the patient, whether a patient has a medical condition. For example, the diagnostic model may be a deep learning ensemble model, a deep neural network (DNN), a convolutional neural networks (CNN), a fully convolutional network (FCN), a recurrent neural network (RCN), a Bayesian network, a graphical probabilistic model, a K-nearest neighbor classifier, a decision forests, a maximum margin method, or the like. The AI models 140-1 through 140-n may be constituent models of the diagnostic model, and may be respectively configured to determine, using medical data of the patient, whether a patient has a medical condition. For example, the AI models 140-1 through 140-n may be trained using different training datasets, such as sub-group training datasets or balanced training datasets, as described in more detail elsewhere herein.

The training device 150 may be configured to train the diagnostic model 130 and the AI models 140-1 through 140-n. For example, the training device 150 may be a server, a computer, a virtual machine, or the like.

The training data database 160 may be configured to store training datasets. For example, the training data database 160 may be a relational database, a distributed database, a cloud database, an object database, a data warehouse, or the like.

The medical data database 170 may be configured to store medical data corresponding to the training data stored in the training data database 160. For example, the medical data database 170 may be a relational database, a distributed database, a cloud database, an object database, a data warehouse, or the like.

The user device 180 may be configured to display information received from the diagnostic platform 120. For example, the user device 180 may be a smartphone, a laptop computer, a desktop computer, a wearable device, a medical device, a radiology device, or the like.

The network 190 may be configured to permit communication between the devices of the system 100. For example, the network 190 may be a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of the devices of the system 100 shown in FIG. 1 are provided as an example. In practice, the system 100 may include additional devices, fewer devices, different devices, or differently arranged devices than those shown in FIG. 1. Additionally, or alternatively, a set of devices (e.g., one or more devices) of the system 100 may perform one or more functions described as being performed by another set of devices of the system 100.

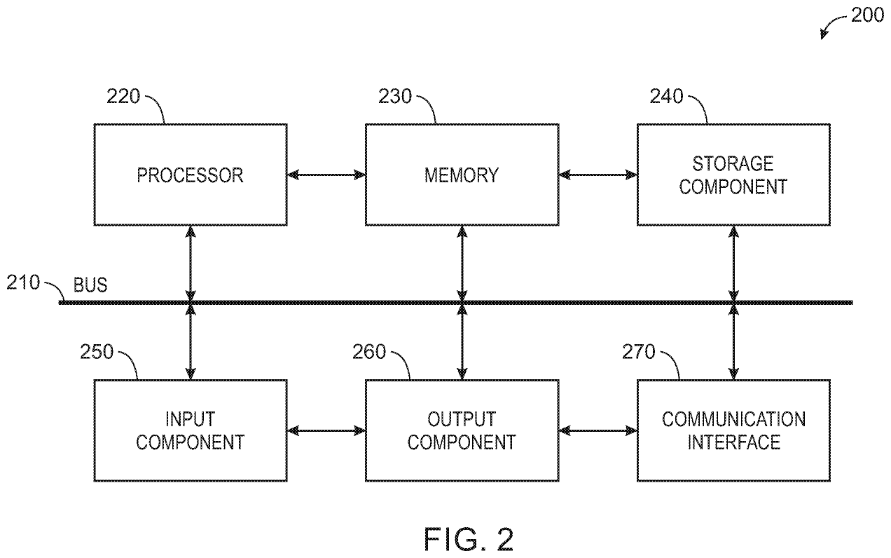

FIG. 2 is a diagram of a device 200 of the system 100 of FIG. 1. The device 200 may correspond to the medical device 110, the diagnostic platform 120, the training device 150, the training data database 160, the medical data database 170, and/or the user device 180. As shown in FIG. 2, the device 200 may include a bus 210, a processor 220, a memory 230, a storage component 240, an input component 250, an output component 260, and a communication interface 270.

The bus 210 includes a component that permits communication among the components of the device 200. The processor 220 may be implemented in hardware, firmware, or a combination of hardware and software. The processor 220 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 220 may include one or more processors 220 configured to perform the operations described herein. For example, a single processor 220 may be configured to perform all of the operations described herein. Alternatively, multiple processors 116220 collectively, may be configured to perform all of the operations described herein, and each of the multiple processors 220 may be configured to perform a subset of the operations descried herein. For example, a first processor 220 may perform a first subset of the operations described herein, a second processor 220 may be configured to perform a second subset of the operations described herein, etc.

The processor 220 may include one or more processors capable of being programmed to perform a function. The memory 230 may include a random access memory (RAM), a read only memory (ROM), and/or another type of dynamic or static storage device (e.g., a flash memory, a magnetic memory, and/or an optical memory) that stores information and/or instructions for use by the processor 220.

The storage component 240 may store information and/or software related to the operation and use of the device 200. For example, the storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 may include a component that permits the device 200 to receive information, such as via user input (e.g., a touch screen display, a keyboard, a keypad, a mouse, a button, a switch, a camera, and/or a microphone). Additionally, or alternatively, the input component 250 may include a sensor for sensing information (e.g., a global positioning system (GPS) component, an accelerometer, a gyroscope, and/or an actuator). The output component 260 may include a component that provides output information from the device 200 (e.g., a display, a speaker for outputting sound at the output sound level, and/or one or more light-emitting diodes (LEDs)).

The communication interface 270 may include a transceiver-like component (e.g., a transceiver and/or a separate receiver and transmitter) that enables the device 200 to communicate with other devices, such as via a wired connection, a wireless connection, or a combination of wired and wireless connections. The communication interface 270 may permit the device 200 to receive information from another device and/or provide information to another device. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The device 200 may perform one or more processes described herein. The device 200 may perform these processes based on the processor 220 executing software instructions stored by a non-transitory computer-readable medium, such as the memory 230 and/or the storage component 240. A computer-readable medium may be defined herein as a non-transitory memory device. A memory device may include memory space within a single physical storage device or memory space spread across multiple physical storage devices.

The software instructions may be read into the memory 230 and/or the storage component 240 from another computer-readable medium or from another device via the communication interface 270. When executed, the software instructions stored in the memory 230 and/or the storage component 240 may cause the processor 220 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the device 200 may include additional components, fewer components, different components, or differently arranged components than those shown in FIG. 2. Additionally, or alternatively, a set of components (e.g., one or more components) of the device 200 may perform one or more functions described as being performed by another set of components of the device 200.

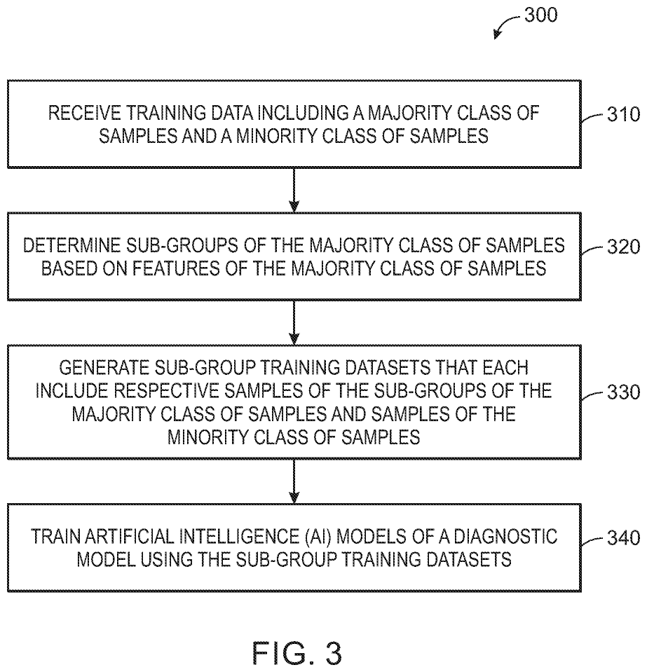

FIG. 3 is a flowchart of an example process 300 for training AI models of a diagnostic model using sub-group training datasets.

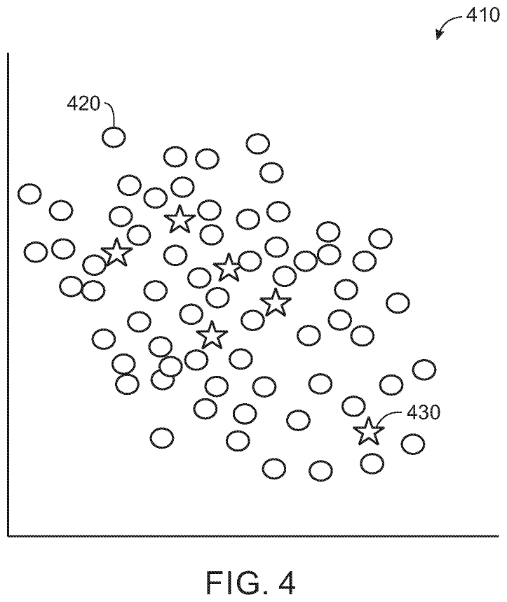

As shown in FIG. 3, the process 300 may include receiving training data including a majority class of samples and a minority class of samples (operation 310). For example, the training device 150 may receive the training data from the training data database 160. The training data may be medical data of patients. For example, the medical data may be ECG data, EEG data, ultrasound data, MRI data, X-ray data, CT data, or the like. Some of the patients may have a medical condition, whereas other patients might not have the medical condition. The medical condition may be any type of underlying medical condition, such as an arrhythmia, a cancer, a lesion, or the like. As an example, if the medical data is ECG data, then the medical condition may be ventricular fibrillation, atrial-paced rhythm, ventricular-paced rhythm, atrial flutter, ectopic atrial tachycardia, sinus bradycardia, sinus tachycardia, junctional bradycardia, atrial fibrillation, left bundle branch block, septal infarct, or the like. The training data may include a majority class of samples. The majority class of samples may be samples of the medical data that constitute a majority of the total number of samples of the training data. For example, the majority class of samples may correspond to medical data of patients that do not have a medical condition. The training data may include a minority class of samples. The minority class of samples may be samples of the medical data that constitute a minority of the total number of samples of the training data. For example, the minority class of samples may correspond to medical data of patients that do have the medical condition. However, in some cases, it should be understood that the majority class of samples may correspond to medical data of patients that do have the medical condition, and the minority class of samples may correspond to medical data of patients that do not have the medical condition. FIG. 4 is a diagram 400 of example training data. As shown in FIG. 4, the training data 410 may include a majority class of samples 420 and a minority class of samples 430. As shown, the training data is “imbalanced.” As used herein, “imbalance” or “balance” may be determined based on a ratio of the cardinality of the majority class of samples to the cardinality of the minority class of samples. For instance, if the training data includes 100 total samples, 90 samples belonging to the majority class, and 10 samples belonging to the minority class, then the ratio may be 9 (i.e., 90/10=9). As used herein, “imbalanced” may refer to a ratio being greater than a threshold (e.g., 5, 6, 7, etc.). Further, as used herein, “balanced” may refer to a ratio being less than a threshold (e.g., 3, 2, 1, etc.).

As further shown in FIG. 3, the process 300 may include determining sub-groups of the majority class of samples based on features of the majority class of samples (operation 320). For example, the training device 150 may determine sub-groups of the majority class of samples based on features of the majority class of samples.

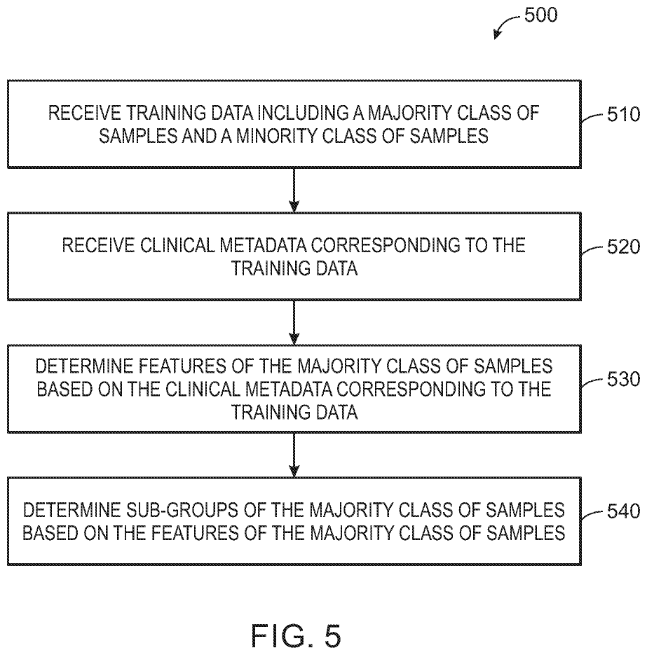

According to an embodiment, the training device 150 may determine sub-groups of the majority class of samples using clinical metadata corresponding to the training dataset. FIG. 5 is a flowchart of an example process 500 for determining sub-groups of a majority class of samples based on features determined in clinical metadata. As shown in FIG. 5, the process 500 may include receiving training data including a majority class of samples and a minority class of samples (operation 510), and receiving clinical metadata corresponding to the training data (operation 520). For example, the training device 150 may receive the training data from the training data database 160, and receive the clinical metadata from the medical data database 170. The clinical metadata may include medical history information, demographic information, treatment information, or the like. As further shown in FIG. 5, the process 500 may include determining features of the majority class of samples based on the clinical metadata corresponding to the training data (operation 530), and determining sub-groups of the majority class of samples based on the features of the majority class of samples (operation 540). For example, the training device 150 may determine features of the majority class of samples based on the clinical metadata corresponding to the training data, and determine sub-groups of the majority class of samples based on the features of the majority class of samples. According to an embodiment, a feature may be any type of feature that permits patients to be grouped based on some commonality. For example, a feature may be age, nationality, occupation, particular medical history, particular treatment history, or the like. A sub-group of the majority class of samples may refer to samples of the majority class of samples that belong to patients that share the feature. For example, a first sub-group may correspond to patients that have cardiac history, a second sub-group may correspond to patients that are “healthy,” etc.

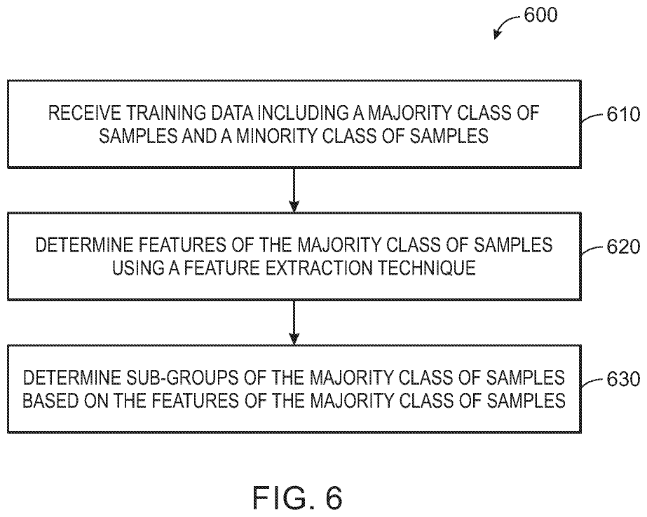

According to an embodiment, the training device 150 may determine sub-groups of the majority class of samples using features extracted from training data. FIG. 6 is a flowchart of an example process 600 for determining sub-groups of a majority class of samples based on features extracted from training data. As shown in FIG. 6, the process 600 may include receiving training data including a majority class of samples and a minority class of samples (operation 610), determining features of the majority class of samples using a feature extraction technique (operation 620), and determining sub-groups of the majority class of samples based on the features of the majority class of samples (operation 630). For example, the training device 150 may receive training data including a majority class of samples and a minority class of samples, determine features of the majority class of samples using a feature extraction technique, and determine sub-groups of the majority class of samples based on the features of the majority class of samples. The feature extraction technique may be a technique that extracts a feature from underlying medical data. For example, if the medical data is ECG data, then the training device 150 may extract features from the ECG data using feature extraction techniques, such as feature extraction algorithms, or the like. According to an embodiment, a feature may be any type of feature that permits patients to be grouped based on some commonality. For example, if the medical data is ECG data, then a feature may be heart rate, cardiac output, stroke volume, cardiac index, system vascular resistance, or the like. In this case, the training device 150 may extract the feature based on an amplitude of a P wave, a duration of the P wave, an amplitude of a Q wave, a duration of the Q wave, an amplitude of an R wave, a duration of the R wave, a PR interval, an amplitude of an S wave, a duration of the S wave, a duration of the QRS complex, an amplitude of a T wave, a duration of the T wave, a QT interval, or the like, determined from the training data.

As further shown in FIG. 3, the process 300 may include generating sub-group training datasets that each include respective samples of the sub-groups of the majority class of samples and samples of the minority class of samples (operation 330). For example, the training device 150 may generate sub-group training datasets that each include respective samples of the sub-groups of the majority class of samples and samples of the minority class of samples.

A sub-group training dataset may include samples of the sub-group of the majority class of samples, and samples of the minority class of samples. According to an embodiment, the sub-group training dataset may include all of the samples of the minority class of samples. Alternatively, the sub-group training dataset may include a subset of samples of the minority class of samples.

A sub-group training dataset may be more balanced than as compared to the training data because the sub-group training dataset includes less samples of the majority class of samples. For instance, if the training data includes 100 total samples, 90 samples belonging to the majority class, 30 samples belonging to a sub-group of the majority class, and 10 samples belonging to the minority class, then the ratio of the training data may be 9 (i.e., 90/10=9) whereas a ratio of the sub-group training dataset may be 3 (i.e., 30/10=3). According to an embodiment, a sub-group training dataset may be entirely balanced by including a same number of samples of the majority class of samples and a number of samples of the minority class of samples. For instance, if the training data includes 100 total samples, 90 samples belonging to the majority class, 30 samples belonging to a sub-group of the majority class, and 10 samples belonging to the minority class, then the ratio of the training data may be 9 (i.e., 90/10=9) whereas a ratio of the sub-group training dataset including 10 samples may be 1 (i.e., 10/10=1).

The training device 150 may generate n sub-group training datasets. According to an embodiment, each of the n sub-group training datasets may be based on a same type of feature. For example, each of the n sub-group training datasets may be based on different cardiac histories, different ages, different nationalities, different health statuses, different treatment histories, or the like. Alternatively, each of the n sub-group training datasets may be based on different types of features. For example, a first sub-group training dataset may be based on age, a second sub-group training dataset may be based on cardiac history, a third sub-group training dataset may be based on treatment history, or the like.

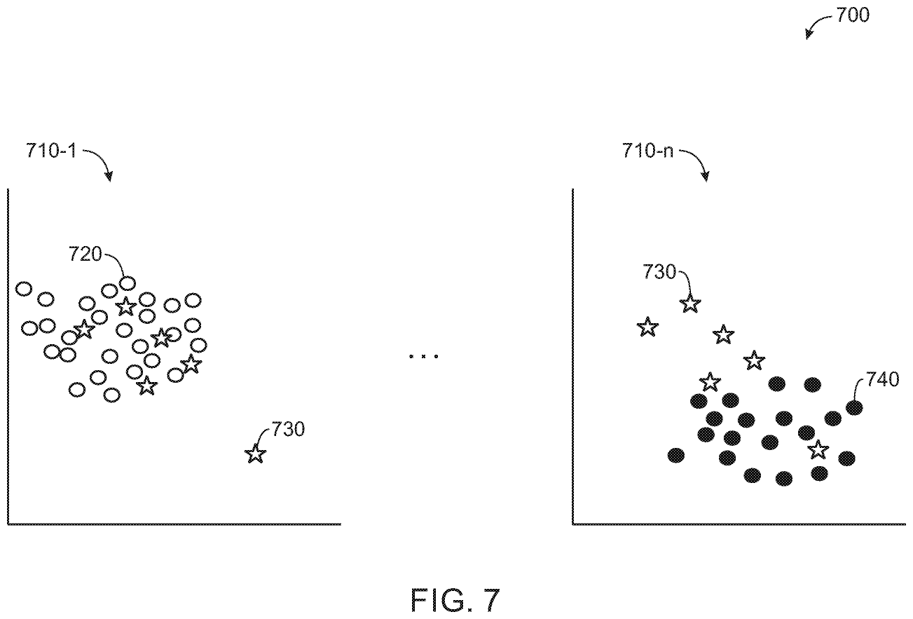

FIG. 7 is a diagram 700 of example sub-group training datasets. As shown in FIG. 7, a first sub-group training dataset 710 may include samples 720 belonging to a first sub-group of the majority class of samples, and samples 730 belonging to the minority class of samples. As further shown in FIG. 7, an n-th sub-group training dataset 710-n may include samples 740 belonging to an n-th sub-group of the majority class of samples, and samples 730 belonging to the minority class of samples.

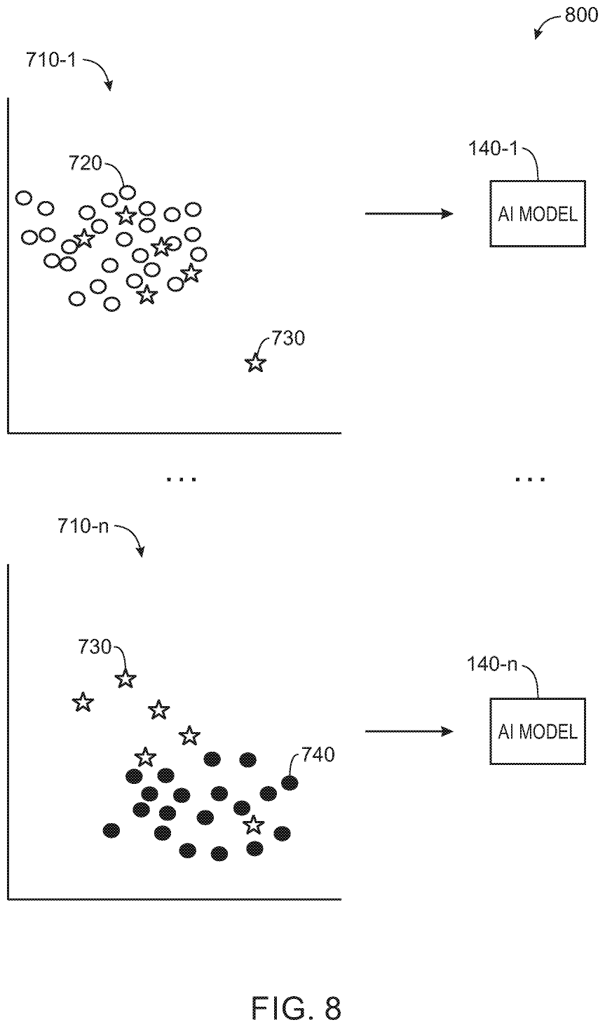

As further shown in FIG. 3, the process 300 may include training AI models of a diagnostic model using the sub-group training datasets (operation 340). For example, the training device 150 may train the AI models 140-1 through 140-n of the diagnostic model 130 using the sub-group training datasets. The training device 150 may train a first AI model 140-1 using a first sub-group training dataset, and may train an n-th AI model 140-n using an n-th sub-group training dataset. FIG. 8 is a diagram 800 of training AI models of a diagnostic model using sub-group training datasets. For example, as shown in FIG. 8, the training device 150 may train a first AI model 140-1 using a first sub-group training dataset 710, and train an n-th AI model 140-n using an n-th sub-group training dataset 710-n.

In this way, the training device 150 may train the AI models 140-1 through 140-n of the diagnostic model 130 using sub-group training datasets that are more balanced than as compared to imbalanced training data and that have constituent samples that share some commonality based on the underlying features on which the sub-group training datasets are grouped. Accordingly, some embodiments herein may provide a diagnostic model 130 and constituent AI models 140-1 through 140-n that are more accurate, that consume less processor resources, that consume less memory resources, that require less training time, that require less ensembles, or the like.

The number and arrangement of the operations of the process 300 shown in FIG. 3 are provided as an example. In practice, the process 500 may include additional operations, fewer operations, different operations, differently ordered, or differently arranged operations than those shown in FIG. 3.

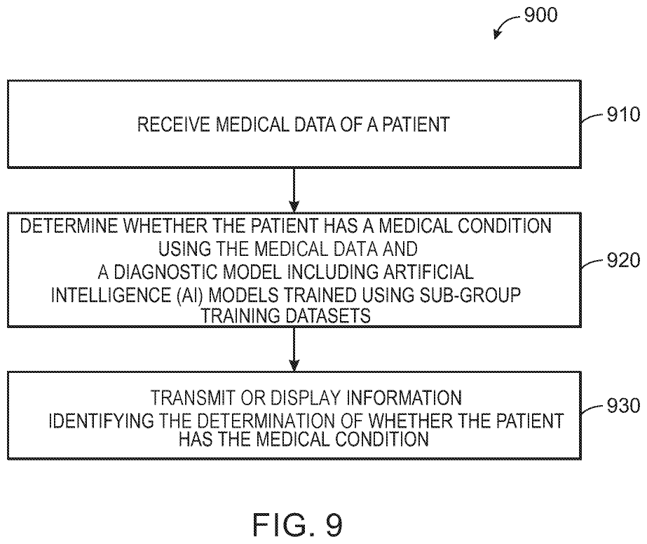

FIG. 9 is a flowchart of an example process 900 for determining whether a patient has a medical condition using a diagnostic model including AI models trained using sub-group training datasets.

As shown in FIG. 9, the process 900 may include receiving medical data of a patient (operation 910). For example, the diagnostic platform 130 may receive medical data of a patient from the medical device 110, the medical data database 170, or the user device 180. The medical data may be ECG data, EEG data, ultrasound data, MRI data, X-ray data, CT data, or the like.

As further shown in FIG. 9, the process 900 may include determining whether the patient has a medical condition using a diagnostic model including AI models trained using sub-group training datasets (operation 920). For example, the diagnostic platform 130 may determine whether the patient has a medical condition using the diagnostic model 130 including the AI models 140-1 through 140-n that are trained using the sub-group training datasets described above in connection FIG. 3. The diagnostic model 130 may be configured to receive the medical data as an input, and provide the medical data to each of the AI models 140-1 through 140-n. The AI models 140-1 through 140-n may each respectively output a determination of whether the patient has the medical condition. The diagnostic model 130 may output a determination based on the respective determinations of the AI models 140-1 through 140-n. According to an embodiment, the diagnostic model may output an uncertainty that is based on the respective determinations of the AI models 140-1 through 140-n. For example, there will be less uncertainty if more of the AI models 140 are in agreement with the determination, and there will be less uncertainty if less of the experts are in agreement with the determination.

As further shown in FIG. 9, the process 900 may include transmitting or displaying information identifying the determination of whether the patient has the medical condition (operation 930). For example, the diagnostic platform 130 may transmit the information identifying the determination of whether the patient has the medical condition to the user device 180 to permit, or cause, the user device 180 to display the information identifying whether the patient has the medical condition. Alternatively, the diagnostic platform 130 may display the information identifying the determination of whether the patient has the medical condition via display.

In this way, the diagnostic model 130 including the AI models 140-1 through 140-n may more accurately determine whether a patient has a medical condition by being trained using sub-group training datasets that are more balanced than as compared to imbalanced training data and that have constituent samples that share some commonality based on the underlying features on which the sub-group training datasets are grouped.

The number and arrangement of the operations of the process 900 shown in FIG. 9 are provided as an example. In practice, the process 500 may include additional operations, fewer operations, different operations, differently ordered, or differently arranged operations than those shown in FIG. 9.

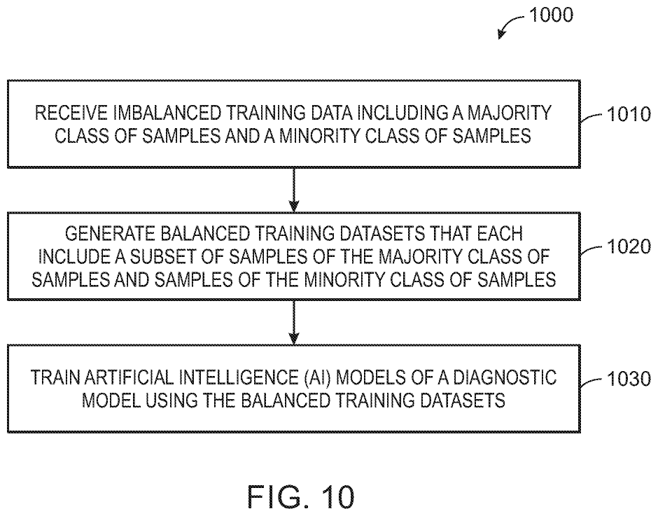

FIG. 10 is a flowchart of an example process 1000 for training AI models of a diagnostic model using balanced training datasets.

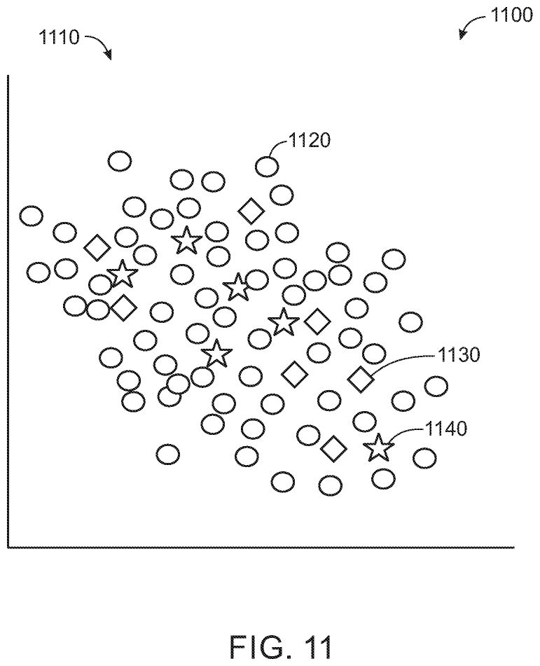

As shown in FIG. 10, the process 1000 may include receiving imbalanced training data including a majority class of samples and a minority class of samples (operation 1010). For example, the training device 150 may receive the imbalanced training data from the training data database 160. The imbalanced training data may be medical data of patients. For example, the medical data may be ECG data, EEG data, ultrasound data, MRI data, X-ray data, CT data, or the like. Some of the patients may have a medical condition, whereas other patients might not have the medical condition. The medical condition may be any type of underlying medical condition, such as an arrhythmia, a cancer, a lesion, or the like. As an example, if the medical data is ECG data, then the medical condition may be ventricular fibrillation, atrial-paced rhythm, ventricular-paced rhythm, atrial flutter, ectopic atrial tachycardia, sinus bradycardia, sinus tachycardia, junctional bradycardia, atrial fibrillation, left bundle branch block, septal infarct, or the like. The training data may include a majority class of samples. The majority class of samples may be samples of the medical data that constitute a majority of the total number of samples of the training data. For example, the majority class of samples may correspond to medical data of patients that do not have a medical condition. The imbalanced training data may include a minority class of samples. The minority class of samples may be samples of the medical data that constitute a minority of the total number of samples of the training data. For example, the minority class of samples may correspond to medical data of patients that do have the medical condition. However, in some cases, it should be understood that the majority class of samples may correspond to medical data of patients that do have the medical condition, and the minority class of samples may correspond to medical data of patients that do not have the medical condition. FIG. 11 is a diagram 1100 of example imbalanced training data. As shown in FIG. 11, the imbalanced training data 1110 may include a majority class of samples 1120, a first minority class of samples 1130, and an n-th minority class of samples 1140. As shown, the training data is “imbalanced” because a ratio between the majority class of samples and the first minority class of samples 1130 and/or a ratio between the majority class of samples and an n-th minority class of samples 1140 is greater than a threshold.

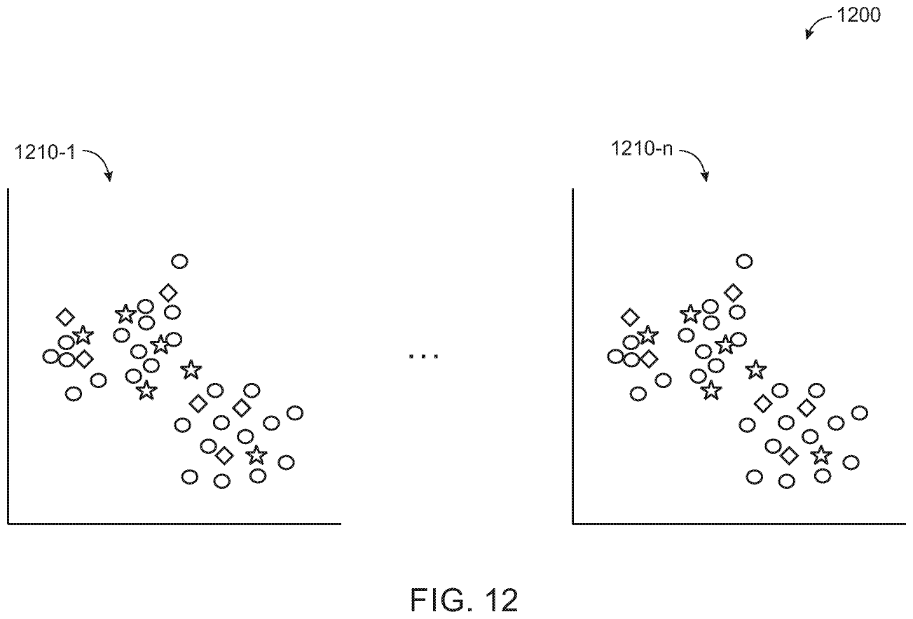

As further shown in FIG. 10, the process 1000 may include generating balanced training datasets that each include a subset of samples of the majority class of samples and samples of the minority class of samples (operation 1020). For example, the training device 150 may generate balanced training datasets that each include a subset of samples of the majority class of samples and samples of the minority class of samples. A balanced training dataset may include samples of the sub-group of the majority class of samples, and samples of the minority class of samples. According to an embodiment, the ratio may be 1. That is, the number of samples of the majority class of samples may be the same as the number of samples of the minority class of samples. Alternatively, the ratio may be less than a threshold, and the number of samples of the majority class of samples may be the different than the number of samples of the minority class of samples. According to an embodiment, the balanced training dataset may include all of the samples of the minority class of samples. Alternatively, the sub-group training dataset may include a subset of samples of the minority class of samples. A balanced training dataset may be more balanced than as compared to the imbalanced training data because the balanced training dataset includes less samples of the majority class of samples. For instance, if the training data includes 100 total samples, 90 samples belonging to the majority class, and 10 samples belonging to the minority class, then the ratio of the imbalanced training data may be 9 (i.e., 90/10=9) whereas a ratio of the balanced training dataset may be 1 (i.e., 10/10=3). FIG. 12 is a diagram 1200 of example balanced training datasets. As shown in FIG. 12, a first balanced training dataset 1210-1 may include less samples 1120 belonging to the majority class of samples as compared to the imbalanced training data 1110, and samples 1130 belonging to the first minority class of samples 1130, and samples 1140 belonging to the n-th minority class of samples 1140. As further shown in FIG. 12, an n-th balanced training dataset 1210-1 may include less samples 1120 belonging to the majority class of samples as compared to the imbalanced training data 1110, and samples 1130 belonging to the first minority class of samples 1130, and samples 1140 belonging to the n-th minority class of samples 1140.

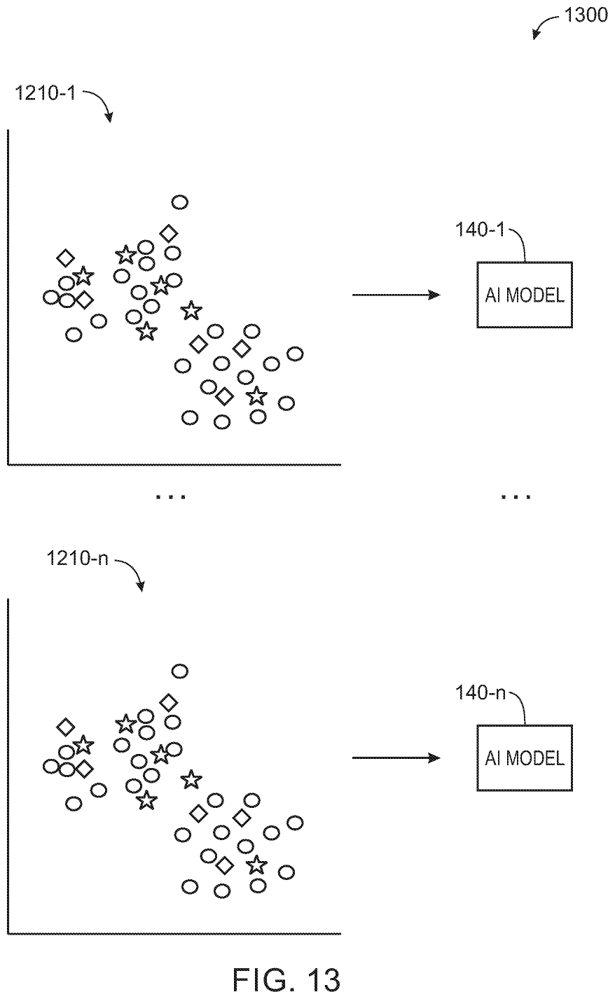

As further shown in FIG. 10, the process 1000 may include training AI models of a diagnostic model using the balanced training datasets (operation 1030). For example, the training device 150 may train the AI models 140-1 through 140-n of the diagnostic model using the balanced training datasets. The training device 150 may train a first AI model 140-1 using a first balanced training dataset, and may train an n-th AI model 140-n using an n-th balanced training dataset. FIG. 13 is a diagram 1300 of training AI models of a diagnostic model using balanced training datasets. For example, as shown in FIG. 13, the training device 150 may train a first AI model 140-1 using a first balanced training dataset 1210-1, and train an n-th AI model 140-n using an n-th balanced training dataset 1210-n.

In this way, the training device 150 may train the AI models 140-1 through 140-n of the diagnostic model 130 using balanced training datasets that are more balanced than as compared to imbalanced training data. Accordingly, some embodiments herein may provide a diagnostic model 130 and constituent AI models 140-1 through 140-n that are more accurate, that consume less processor resources, that consume less memory resources, that require less training time, that require less ensembles, or the like.

The number and arrangement of the operations of the process 1000 shown in FIG. 10 are provided as an example. In practice, the process 1000 may include additional operations, fewer operations, different operations, differently ordered, or differently arranged operations than those shown in FIG. 10.

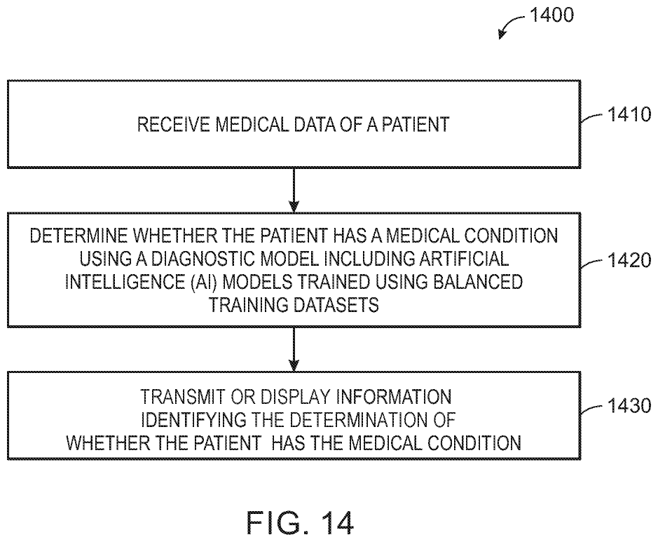

FIG. 14 a flowchart of an example process 1400 for determining whether a patient has a medical condition using a diagnostic model including AI models trained using balanced training datasets.

As shown in FIG. 14, the process 1400 may include receiving medical data of a patient (operation 1410). For example, the diagnostic platform 120 may receive medical data of a patient from the medical device 110, the medical data database 170, or the user device 180. The medical data may be ECG data, EEG data, ultrasound data, MRI data, X-ray data, CT data, or the like.

As further shown in FIG. 14, the process 1400 may include determining whether the patient has a medical condition using a diagnostic model including AI models trained using balanced training datasets (operation 1420). For example, the diagnostic platform 130 may determine whether the patient has a medical condition using the diagnostic model 130 including the AI models 140-1 through 140-n that are trained using the balanced training datasets described above in connection FIG. 10. The diagnostic model 130 may be configured to receive the medical data as an input, and provide the medical data to each of the AI models 140-1 through 140-n. The AI models 140-1 through 140-n may each respectively output a determination of whether the patient has the medical condition. The diagnostic model 130 may output a determination based on the respective determinations of the AI models 140-1 through 140-n. According to an embodiment, the diagnostic model may output an uncertainty that is based on the respective determinations of the AI models 140-1 through 140-n. For example, there will be less uncertainty if more of the AI models 140 are in agreement with the determination, and there will be less uncertainty if less of the experts are in agreement with the determination.

As further shown in FIG. 14, the process 1400 may include providing information identifying whether the patient has the medical condition (operation 1430). For example, the diagnostic platform 130 may transmit the information identifying the determination of whether the patient has the medical condition to the user device 180 to permit, or cause, the user device 180 to display the information identifying whether the patient has the medical condition. Alternatively, the diagnostic platform 130 may display the information identifying the determination of whether the patient has the medical condition via display.

In this way, the diagnostic model 130 including the AI models 140-1 through 140-n may more accurately determine whether a patient has a medical condition by being trained using balanced training datasets that are more balanced than as compared to imbalanced training data.

The number and arrangement of the operations of the process 1400 shown in FIG. 14 are provided as an example. In practice, the process 500 may include additional operations, fewer operations, different operations, differently ordered, or differently arranged operations than those shown in FIG. 14.

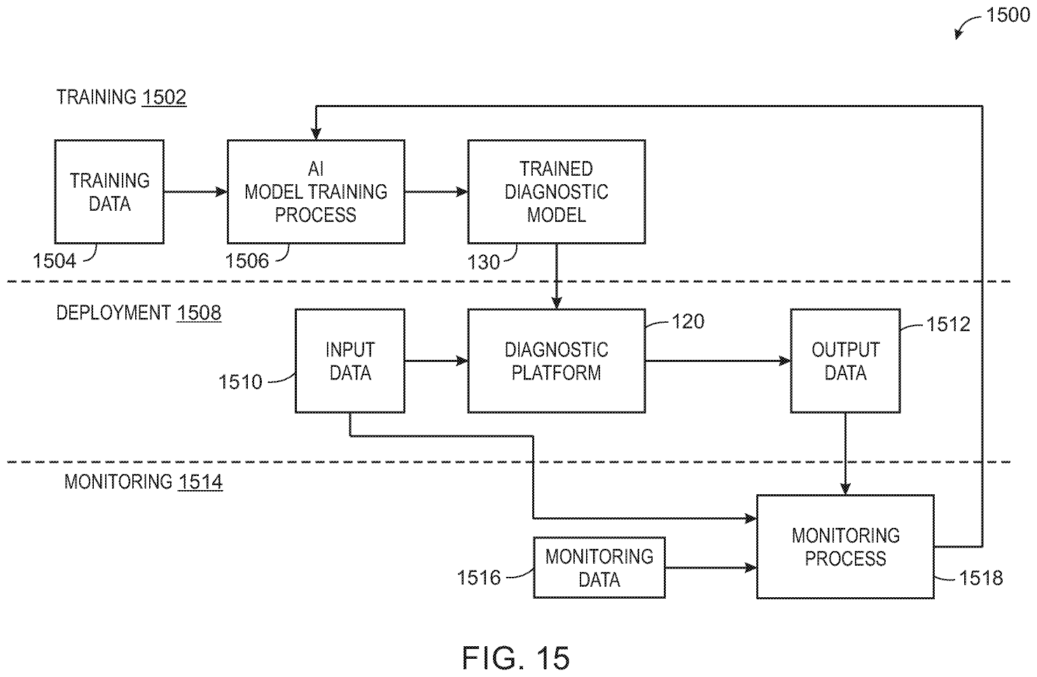

FIG. 15 is a diagram 1500 of an example process for training, deploying, and monitoring a diagnostic model. The system 100 may generate, store, train, and/or use diagnostic model 130 including the AI models 140-1 through 140-n. According to an embodiment, the system 100 may include the diagnostic model 130 including the AI models 140-1 through 140-n and/or instructions associated with the diagnostic model 130 including the AI models 140-1 through 140-n. For example, the system 100 may include instructions for generating the diagnostic model 130 including the AI models 140-1 through 140-n, training the diagnostic model 130 including the AI models 140-1 through 140-n, using the diagnostic model 130 including the Al models 140-1 through 140-n, etc. According to another embodiment, a system or device other than the system 100 may be used to generate and/or train the diagnostic model 130 including the AI models 140-1 through 140-n. For example, a system or device may include instructions for generating the diagnostic model 130 including the AI models 140-1 through 140-n, and/or instructions for training the diagnostic model 130 including the AI models 140-1 through 140-n. The system or device may provide a resulting trained diagnostic model 130 including the AI models 140-1 through 140-n to the system 100 for use.

As shown in FIG. 15, according to an embodiment, the process 1500 may include a training phase 1502, a deployment phase 1508, and a monitoring phase 1514. In the training phase 1502, at operation 1506, the process 1500 may include receiving and processing training data 1504 to generate a trained diagnostic model 130 including the AI models 140-1 through 140-n for performing one or more operations of any one of the processes 900 or 1400. The training data 1004 may include the sub-group training datasets and/or the balanced training datasets.

Generally, the diagnostic model 130 including the AI models 140-1 through 140-n may include a set of variables (e.g., nodes, neurons, filters, or the like) that are tuned (e.g., weighted, biased, or the like) to different values via the application of the training data 1504. According to an embodiment, the training process at operation 1506 may employ supervised, unsupervised, semi-supervised, and/or reinforcement learning processes to train the diagnostic model 130 including the Al models 140-1 through 140-n. According to an embodiment, a portion of the training data 1504 may be withheld during training and/or used to validate the trained diagnostic model 130 including the AI models 140-1 through 140-n.

For supervised learning processes, the training data 1504 may include labels or scores that may facilitate the training process by providing a ground truth. The diagnostic model 130 including the AI models 140-1 through 140-n may have variables set at initialized values (e.g., at random, based on Gaussian noise, based on pre-trained values, or the like). The diagnostic model 130 including the AI models 140-1 through 140-n may provide an output, and the output may be compared with the corresponding label or score (e.g., the ground truth), which may then be back-propagated through the diagnostic model 130 including the AI models 140-1 through 140-n to adjust the values of the variables. This process may be repeated for a plurality of samples at least until a determined loss or error is below a predefined threshold. According to an embodiment, some of the training data 1504 may be withheld and used to further validate or test the trained diagnostic model 130 including the AI models 140-1 through 140-n.

For unsupervised learning processes, the training data 1504 may not include pre-assigned labels or scores to aid the learning process. Instead, unsupervised learning processes may include clustering, classification, or the like, to identify naturally occurring patterns in the training data 1504. As an example, the training data 1504 may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data 1504 with pre-assigned labels or scores and training data 1504 without pre-assigned labels or scores may be used to train the diagnostic model 130 including the AI models 140-1 through 140-n.

When reinforcement learning is employed, an agent (e.g., an algorithm) may be trained to make a determination regarding whether a patient has a medical condition from the training data 1504 through trial and error. For example, based on making a determination, the agent may then receive feedback (e.g., a positive reward if the determination was above a predetermined threshold), adjust its next decision to maximize the reward, and repeat until a loss function is optimized.

After being trained, the diagnostic model 130 including the AI models 140-1 through 140-n may be stored and subsequently applied by system 100 during the deployment phase 1508. For example, during the deployment phase 1508, the trained diagnostic model 130 including the AI models 140-1 through 140-n executed by the system 100 may receive input data 1510 for performing one or more operations of any one of processes 900 or 1400. The input data 1510 may be medical data of a patient.

After being applied by system 100 during the deployment phase 1508, the trained diagnostic model 130 including the AI models 140-1 through 140-n may be monitored during the monitoring phase 1514. The monitoring data 1516 may include data that is output by the diagnostic model 130 including the AI models 140-1 through 140-n. During the monitoring process 1518, the monitoring data 1516 may be analyzed along with the determined output data 1512 and input data 1510 to determine an accuracy of the trained diagnostic model 130 including the AI models 140-1 through 140-n. According to an embodiment, based on the analysis, the process 1500 may return to the training phase 1502, where at operation 1506 values of one or more variables of the trained diagnostic model 130 including the AI models 140-1 through 140-n may be adjusted to improve the accuracy of the diagnostic model 130 including the AI models 140-1 through 140-n.

The example process 1500 described above is provided merely as an example, and may include additional, fewer, different, or differently arranged aspects than depicted in FIG. 15.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A method comprising:
receiving medical data of a patient;
determining whether the patient has a medical condition using the medical data and a diagnostic model including artificial intelligence (AI) models; and
transmitting or displaying information identifying the determination of whether the patient has the medical condition,
wherein the diagnostic model including the AI models is trained by:
receiving training data including a majority class of samples corresponding to medical data of patients that do not have the medical condition and a minority class of samples corresponding to medical data of patients that do have the medical condition;
determining sub-groups of the majority class of samples based on features of the majority class of samples;
generating sub-group training datasets that each include respective samples of the sub-groups of the majority class of samples and samples of the minority class of samples; and
training the AI models of the diagnostic model using the sub-group training datasets.

2. The method of claim 1, wherein the features are determined using clinical metadata associated with the majority class of samples.

3. The method of claim 1, wherein the features are determined by extracting the features from the training data using a feature extraction technique.

4. The method of claim 1, wherein the AI models are deep learning ensemble models.

5. The method of claim 1, wherein each sub-group training dataset is based on a same type of feature.

6. The method of claim 1, wherein each sub-group training dataset is based on a different type of feature.

7. The method of claim 1, wherein a ratio between a number of samples of the majority class of samples and a number of samples of the minority class of samples for each of the sub-group training datasets is less than a ratio between a number of samples of the majority class of samples and a number of samples of the minority class of samples for the training data.

8. A device comprising:
a memory configured to store instructions; and
one or more processors configured to execute the instructions to perform operations comprising:
receiving medical data of a patient;
determining whether the patient has a medical condition using the medical data and a diagnostic model including artificial intelligence (AI) models; and
transmitting or displaying information identifying the determination of whether the patient has the medical condition,
wherein the diagnostic model including the AI models is trained by:
receiving training data including a majority class of samples corresponding to medical data of patients that do not have the medical condition and a minority class of samples corresponding to medical data of patients that do have the medical condition;
determining sub-groups of the majority class of samples based on features of the majority class of samples;
generating sub-group training datasets that each include respective samples of the sub-groups of the majority class of samples and samples of the minority class of samples; and
training the AI models of the diagnostic model using the sub-group training datasets.

9. The device of claim 8, wherein the features are determined using clinical metadata associated with the majority class of samples.

10. The device of claim 8, wherein the features are determined by extracting the features from the training data using a feature extraction technique.

11. The device of claim 8, wherein the AI models are deep learning ensemble models.

12. The device of claim 8, wherein each sub-group training dataset is based on a same type of feature.

13. The device of claim 8, wherein each sub-group training dataset is based on a different type of feature.

14. The device of claim 8, wherein a ratio between a number of samples of the majority class of samples and a number of samples of the minority class of samples for each of the sub-group training datasets is less than a ratio between a number of samples of the majority class of samples and a number of samples of the minority class of samples for the training data.

15. A non-transitory computer-readable medium storing instructions that, when executed by one or more processors, cause the one or more processors to perform operations comprising:
receiving medical data of a patient;
determining whether the patient has a medical condition using the medical data and a diagnostic model including artificial intelligence (AI) models; and
transmitting or displaying information identifying the determination of whether the patient has the medical condition,
wherein the diagnostic model including the AI models is trained by:
receiving training data including a majority class of samples corresponding to medical data of patients that do not have the medical condition and a minority class of samples corresponding to medical data of patients that do have the medical condition;
determining sub-groups of the majority class of samples based on features of the majority class of samples;
generating sub-group training datasets that each include respective samples of the sub-groups of the majority class of samples and samples of the minority class of samples; and
training the AI models of the diagnostic model using the sub-group training datasets.

16. The non-transitory computer-readable medium of claim 15, wherein the features are determined using clinical metadata associated with the majority class of samples.

17. The non-transitory computer-readable medium of claim 15, wherein the features are determined by extracting the features from the training data using a feature extraction technique.

18. The non-transitory computer-readable medium of claim 15, wherein the AI models are deep learning ensemble models.

19. The non-transitory computer-readable medium of claim 15, wherein each sub-group training dataset is based on a same type of feature or wherein each sub-group training dataset is based on a different type of feature.

20. The non-transitory computer-readable medium of claim 15, wherein a ratio between a number of samples of the majority class of samples and a number of samples of the minority class of samples for each of the sub-group training datasets is less than a ratio between a number of samples of the majority class of samples and a number of samples of the minority class of samples for the training data.
